(19) United States
(12) Patent Application Publication
(10) Pub. No.: US 2004/0170286 A1
(43) Pub. Date: Sep. 2, 2004

(54) Method for controlling an acoustic system in a vehicle

(75) Inventors: Stephan Durach, Muenchen (DE); Danny Borke, Muenchen (DE); Alexander Augst, Muenchen (DE)

Correspondence Address: CROWELL & MORING LLP, INTELLECTUAL PROPERTY GROUP, P.O. BOX 14300, WASHINGTON, DC, 20044-4300, US

(73) Assignee: Bayerische Motoren Werke Aktiengesellschaft

(21) Appl. No.: 10/784,190

(22) Filed: Feb. 24, 2004

(30) Foreign Application Priority Data
Feb. 27, 2003 (DE) 10308414.2

(51) Int. Cl. A61F 11/06; G10K 11/16; H03B 29/00

(52) U.S. Cl. 381/71.4; 381/86

(57) ABSTRACT

A method of controlling an acoustic system in a vehicle, in which the interior is monitored by an interior sensing system, at least the position of the occupant's head in the interior is recognized by an object recognition system on the basis of the data supplied by the interior sensing system, and a setting of the acoustic system that is optimized for the occupants is performed automatically by a control unit as a function of seat occupancy and the position of the occupant's head in the interior.

This application claims the priority of German application 10308414.2-34, filed Feb. 27, 2003, the disclosure of which is expressly incorporated by reference herein.

### BACKGROUND AND SUMMARY OF THE INVENTION

This invention relates to a method of controlling an acoustic system in a vehicle.

It is known that the audio settings in motor vehicles can be made automatically as a function of a person who has been identified.

For example, German Patent 100 18 652 A1 describes a monitoring system using a camera that can be used for face recognition. This makes it possible to automatically select the favorite car radio station of the occupant recognized.

There are also known car radios in which the sound balance can be adjusted manually. This function must be changed each time there is a change in the number of people in the vehicle, so that optimized sound is achieved for all occupants.

In addition, there are known active acoustic damping devices such as that known from German Patent 33 42 928 A1 which also permit a reduction in noise interference at a certain location, referred to below as the active site. The rigid active sites at which a reduction in noise is possible are relatively small, so it may happen that when a passenger in the vehicle moves, he/she comes to be partially outside of the active site.

The object of this invention is to improve upon the methods already known for controlling an acoustic system.

This object is achieved according to this invention by a method of controlling an acoustic system in a vehicle in which the interior is monitored by an interior sensing system, at least the position of the occupant's head in the interior is recognized by an object recognition system from the data supplied by the interior sensing system, and acoustic system settings that are optimized for the occupants are automatically made by a control unit as a function of the seat occupancy and the position of the head(s) of the occupant(s) in the interior.

The new statutory regulation pertaining to airbag systems in the United States (FMVSS208 from NHTSA) creates the necessity for an interior sensing system for determining seat occupancy and the position of the passenger's head. For many premium-class automobiles, an interior camera system, usually a 3D camera system, is provided for this purpose.

It is advantageous to also use this interior sensing system, which is already present, preferably an interior camera system, to control an acoustic system. Therefore, there is no additional cost for the camera system for the automobile manufacturer.

### DETAILED DESCRIPTION

The acoustic system comprises all the acoustic reception and transmission components in the vehicle. Transmission components for acoustic playback include, for example, the loudspeaker of the car radio. Reception components for acoustic pickup include, for example, the microphones of the car telephone or the voice control system. In playback by loudspeaker, signal output is controlled by the position of the occupant's head, and in pickup by microphone, a sound analysis is performed after the signal is received, this analysis being controlled by the occupant's head position.

Due to the inventive method, the occupants of a vehicle need not concern themselves about an optimized setting of the acoustic system. The interior sensing system and the object recognition system recognize the occupancy of the seats by persons and the positions of their heads in the interior. This method is used to adjust the sound in the vehicle interior so that the best possible sound is obtained for each occupant. On the other hand, the inventive method offers optimized voice reception for the reception components. If the position of an occupant's head in the vehicle interior changes or if an occupant leaves the vehicle, the settings of the acoustic system are automatically adapted to the corresponding changes.

For example, if there are two people on the front seats of a motor vehicle and they want to listen to music from the car radio, the two loudspeakers in the front part are triggered approximately equally for a uniformly distributed balance. Therefore, the two occupants have approximately equally good sound and the best possible sound corresponding to the given circumstances. If one person leaves the vehicle, the two loudspeakers are controlled so that optimized sound is achieved for the sole occupant, depending on the position of his head in the interior.

The interior sensing system including the object recognition system and the control unit may be installed in a joint control device either separately or combined with other systems.

An advantageous embodiment is offered by the inventive method when the optimized setting of the acoustic system is performed by the control unit as a function of the position of the head of at least one occupant.

The position of the head also includes, for example, the inclination of the head and turning of the head. This embodiment is especially suitable when there is only one occupant in the vehicle. The inclination or turning of the head is determined, for example, from the angle of the head with respect to the longitudinal axis of the vehicle and/or the angle of the head with respect to the transverse axis and/or the angle of the head with respect to the vertical axis. The inclination and/or turning of the head, which is monitored in realtime, may be used for optimized adaptation of the amplitude and phase of the signals from various loudspeakers in the vehicle. The amplitude balance between the loudspeakers may thus be implemented for optimum binaural sound reception and for various three-dimensional sound effects.

This method is particularly advantageous when active noise suppression is performed by the control unit according to this invention, so that the active site of the noise suppression follows the current position of the occupant's head.

A means of active noise suppression is already known. With the inventive method, however, it is possible for the active site at which the noise is to be suppressed to be variable. The object recognition system detects the positions of the occupants' heads and at the same time the head positions thus detected represent the center of the active site for the noise suppression. Noise is detected at the active site by a microphone system and is reduced by methods that are already known for active noise suppression. Therefore, the occupant(s) is/are always at the center of the noise suppression, thus permitting satisfactory enjoyment of music, for example.

Sounds from the exterior of the vehicle (e.g., the honking of other vehicles), which must be received according to statutory requirements, can be piped in from the exterior through the acoustic system of the vehicle, for example. This results in the possibility of designing the noise suppression system exclusively for suppressing the inherent noise in the vehicle.

In addition, a separate sound source may be supplied to each occupant. In this case, independent active sites with separate sound from independent sound sources are made available to different occupants at the same time.

This inventive embodiment can also be used in conjunction with a hands-free device or a voice control system. Depending on the current position of an occupant's head, a stereo microphone system of the hands-free device according to known methods will filter out and analyze only the noise coming from the direction of the occupants (on the basis of the amplitude and phase of the signals arriving at the various microphones with the help of digital signal processing). The noise reduction is achieved here by restricting the solid angle from which the useful sound (speech) is coming. This improves the detected speech quality even if the person speaking changes the position of his head while speaking. The vehicle noise and other interfering noise from the interior and exterior of the vehicle can thus be filtered out to a considerable extent. This application is especially advantageous for systems based on voice recognition.

The inventive control is turned on and off either arbitrarily by an operating element or automatically. In addition, it is possible to identify the various occupants, preferably the driver of the vehicle, and to differentiate him from the others by means of the interior sensing system and object recognition system in order to automatically retrieve an audio setting, i.e., an acoustic setting, that has already been stored as a function of the person identified. Then there is only the precision adjustment of the acoustic system to be performed according to the inventive method.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1. Method for controlling an acoustic system in a vehicle, in which the interior is monitored by an interior sensing system, at least the position of an occupant's head in the interior is recognized by an object recognition system from the data supplied by the interior sensing system and a setting of the acoustic system that is optimized for the occupant is performed automatically by a control unit as a function of seat occupancy and the position of the occupant's head in the interior.

2. Method as claimed in claim 1, wherein the optimized setting of the acoustic system is performed by the control unit as a function of the position of the head of at least one of a plurality of occupants.

3. Method as claimed in claim 1, wherein active noise suppression is performed by the control unit so that the active site of the noise suppression follows an instantaneous position of the occupant's head.

4. Method as claimed in claim 2, wherein active noise suppression is performed by the control unit so that the active site of the noise suppression follows an instantaneous position of at least one of the heads of the plurality of occupants.

5. A method for controlling an acoustic system in a vehicle, comprising the steps of:
monitoring an interior of the vehicle with an interior sensing system;
recognizing at least a position of at least one interior occupant's head with an object recognition system using data supplied by the interior sensing system; and
optimizing a setting of the acoustic system automatically with a control unit as a function of interior occupancy and the position of the at least one occupant's head in the interior.

6. The method of claim 5,.wherein in the step of optimized the acoustic system setting is performed by the control unit as a function of the position of the head of at least one of a plurality of occupants.

7. The method of claim 5, wherein the control unit performs an active noise suppression such that an active site of the noise suppression follows an instantaneous position of the occupant's head.

8. The method of claim 6, wherein active noise suppression is performed by the control unit so that the active site of the noise suppression follows an instantaneous position of at least one of the heads of the plurality of occupants.
